(19) United States
(12) Patent Application Publication
(10) Pub. No.: US 2018/0085066 A1
(43) Pub. Date: Mar. 29, 2018

(54) PULSATION MEASURING APPARATUS, LIGHT INTENSITY CONTROL METHOD, AND PROGRAM

(71) Applicant: Renesas Electronics Corporation, Tokyo (JP)

(72) Inventor: Masaki JINNOUCHI, Tokyo (JP)

(21) Appl. No.: 15/669,849

(22) Filed: Aug. 4, 2017

(30) Foreign Application Priority Data
Sep. 29, 2016 (JP) 2016-191708

(51) Int. Cl. A61B 5/00; G01P 13/00; G01J 1/44; A61B 5/024

(52) U.S. Cl. CPC A61B 5/721; G01P 13/00; A61B 2562/0219; A61B 5/02416; G01J 1/44

(57) ABSTRACT

There is a need to be able to perform optical calibration according to changes in measurement situations after the start of measurement.

An LED 21 irradiates light to a subject. An optical detector 22 outputs an optical detection signal. A signal quality calculation portion 14 calculates signal quality of a pulsation signal generated based on the optical detection signal. A body motion level determination portion 15 determines whether the subject maintains a quiescent state based on acceleration detected by an acceleration sensor. A light intensity determination portion 16 determines luminescence quantity of the LED 21 based on the signal quality of the pulsation signal when the subject is determined to maintain a quiescent state.

### CROSS-REFERENCE TO RELATED APPLICATIONS

The disclosure of Japanese Patent Application No. 2016-191708 filed on Sep. 29, 2016 including the specification, drawings and abstract is incorporated herein by reference in its entirety.

### BACKGROUND

The present intention relates to a pulsation measuring apparatus that irradiates light to a subject and generates pulsation information, for example.

The present intention relates to a light intensity control method and a program in the above-mentioned pulsation measuring apparatus.

There is known a pulsation measuring apparatus (pulse monitor) using a pulsation sensor that uses a light generator such as an LED (light emitting diode) and an optical detector such as a phototransistor or a photodiode. As a related art, Japanese Unexamined Patent Application Publication No. 2016-86873 describes a vital sensor module capable of measuring the pulsation. The vital sensor module described in Japanese Unexamined Patent Application Publication No. 2016-86873 includes a light-emitting element placed on the surface of a substrate and a light-sensitive element placed on the surface of the substrate separately from the light-emitting element.

The pulse wave detection sensitivity is subject to individual differences. The luminescence quantity of the light-emitting element needs to be varied depending on a subject being tested in order to acquire a substantially constant output (pulse wave amplitude). Japanese Unexamined Patent Application Publication No. 2016-86873 describes that optical calibration is performed before pulse wave measurement to optimize the luminescence quantity of the light-emitting element during the pulse wave measurement. The optical calibration monitors an output from the light-sensitive element while varying the luminescence quantity of the light-emitting element from a relatively low state to a relatively high state to determine a luminescence quantity optimized for the subject being tested.

### SUMMARY

According to Japanese Unexamined Patent Application Publication No. 2016-86873, however, the optical calibration is performed only once before the measurement. The luminescence quantity of the light-emitting element is fixed to the luminescence quantity determined by the optical calibration while pulse waves are measured subsequently. The Japanese Unexamined Patent Application Publication No. 2016-86873 therefore leaves an issue of unsuccessfully performing the optical calibration depending on variations in measurement situations after the measurement starts.

These and other objects and novel features may be readily ascertained by referring to the following description of the present specification and appended drawings.

According to an embodiment, a pulsation measuring apparatus and a light intensity control method determine whether a subject maintains a quiescent state, based on acceleration detected by an acceleration sensor and, when the subject is determined to maintain a quiescent state, control the light intensity of light irradiated to the subject based on signal quality of a pulsation signal.

According to an embodiment, a program allows a processor to determine whether a subject maintains a quiescent state, based on acceleration detected by an acceleration sensor and, when the subject is determined to maintain a quiescent state, control the light intensity of light irradiated to the subject based on signal quality of a pulsation signal.

According to the above-mentioned embodiment, a pulsation measuring apparatus, a light intensity control method, and a program can adjust the luminescence quantity of a light-emitting portion even after the start of measurement.

### BRIEF DESCRIPTION OF THE DRAWINGS

FIG. 1 is a block diagram illustrating a pulsation measuring apparatus according to an embodiment;

FIG. 2 is a flowchart illustrating an operation procedure of optical calibration when measurement starts;

FIG. 3 is a graph illustrating example optical detection signals output from an optical detector;

FIG. 4 is a diagram illustrating an example of configuring saturation alarm ranges;

FIG. 5 is a flowchart illustrating an operation procedure of optical calibration performed during pulsation measurement; and

FIG. 6 is a diagram illustrating operating waveforms of respective portions during pulsation measurement.

### DETAILED DESCRIPTION

Prior to explanation of the embodiment, the background to the development of the following embodiment will be described. Optical calibration before the measurement is performed according to the following procedure, for example. Immediately after the calibration starts, the luminescence quantity of the light-emitting element is set to a default value such as a maximum value. The luminescence quantity of the light-emitting element gradually decreases each time the light-emitting element blinks. As the luminescence quantity decreases, a detection signal for reflected light detected in the light-sensitive element recovers from a saturation state. The luminescence quantity is fixed when an AD value of a ΔΣAD converter to perform AD (Analog to Digital) conversion on detection signals for the reflected light reaches a center value (e.g., value 0).

Normally, the ΔΣAD converter is configurationally preceded by a PGA (Programmable Gain Amplifier) that amplifies a signal and adjusts a signal level. A reference voltage supplied to the PGA is used to adjust the level of a signal input to the ΔΣAD converter. In the above-mentioned optical calibration, the reference voltage supplied to the PGA is fixed to the default value (specified value) until the calibration is completed.

However, the inventors noticed that the above-mentioned optical calibration involves the following issues. As a first issue, the optical calibration fixes the luminescence quantity of the light-emitting element. The luminescence quantity of the light-emitting element remains unchanged even if the measurement situation changes afterward. As a second issue, a risk of saturation increases due to the reflected light caused by entry of the outside light being used or sweating when a default value of the PGA reference voltage approximates to the maximum value. The default value of the reference voltage needs to be set to be relatively low by providing a margin.

As a third issue, the above-mentioned optical calibration terminates when a ΔΣAD value reaches the center value even just once. The luminescence quantity is unstable even on the same person. As a fourth issue, the luminescence quantity tends to be large when the optical calibration gradually decreases the luminescence quantity. This setting is easily saturated and is unfavorable depending on situations. As a fifth issue, the ΔΣAD value needs to be monitored to adjust the luminescence quantity. The time of two to four seconds is required until the value is fixed. As a sixth issue, the luminescence quantity of the light-emitting element starts from the same value. This does not cover individual difference such as white or black color and induces many cases that cannot be measured easily.

With reference to the accompanying drawings, the description below explains in detail an embodiment that uses means to solve at least one of the above-mentioned issues. The description and drawings are omitted and simplified as needed in order to clarify the explanation. Each element illustrated in the drawings as a function block to perform various processes can be configured as hardware including a CPU (Central Processing Unit), memory, and other circuits and can be embodied as software including a program loaded into the memory. It is therefore understood by those skilled in the art that the function blocks can be embodied as hardware only, as software only, or as combinations of these and are not limited to any thereof. In the drawings, mutually corresponding elements are designated by the same reference symbols and a duplicate explanation is omitted as needed.

The above-mentioned program is stored by using various types of non-transitory computer readable medium and can be supplied to computers. The non-transitory computer readable medium includes various types of tangible storage medium. Examples of the non-transitory computer readable medium include magnetic recording media (e.g., flexible disks, magnetic tape, and hard disks), optical magnetic recording media (e.g., optical magnetic disks), CD-ROM (Read Only Memory), CD-R, CD-R/W, and semiconductor memory (e.g., mask ROM, ROM, PROM (Programmable ROM), EPROM (Erasable PROM), flash ROM, and RAM (Random Access Memory)). The program may be supplied to computers through various types of transitory computer readable medium. Examples of the transitory computer readable medium include electric signals, optical signals, and electromagnetic waves. The transitory computer readable medium can supply the program to computers via wired communication paths such as electric wires and optical fibers or wireless communication paths.

The description below may divide the embodiment into a plurality of sections or embodiments as needed. Unless explicitly specified, the divisions are not unrelated to each other. One provides a modification, an application, a detailed explanation, or a supplementary explanation about all or part of the others. The number of elements (including the number of items, values, quantities, and ranges) referred to in the following embodiment is not limited to a specific value and may be greater or smaller than or equal to the specific value except the number of elements is explicitly specified or is obviously limited to the specific value in principle.

Constituent elements (including operation steps) of the following embodiment are not required unless explicitly specified or obviously required in principle. Similarly, shapes or positional relation of the constituent elements referred to in the following embodiment include those substantially approximate or similar to the shapes unless explicitly specified and obviously considered different in principle. The same applies to the above-mentioned number of elements (including the number of items, values, quantities, and ranges).

### [Configuration]

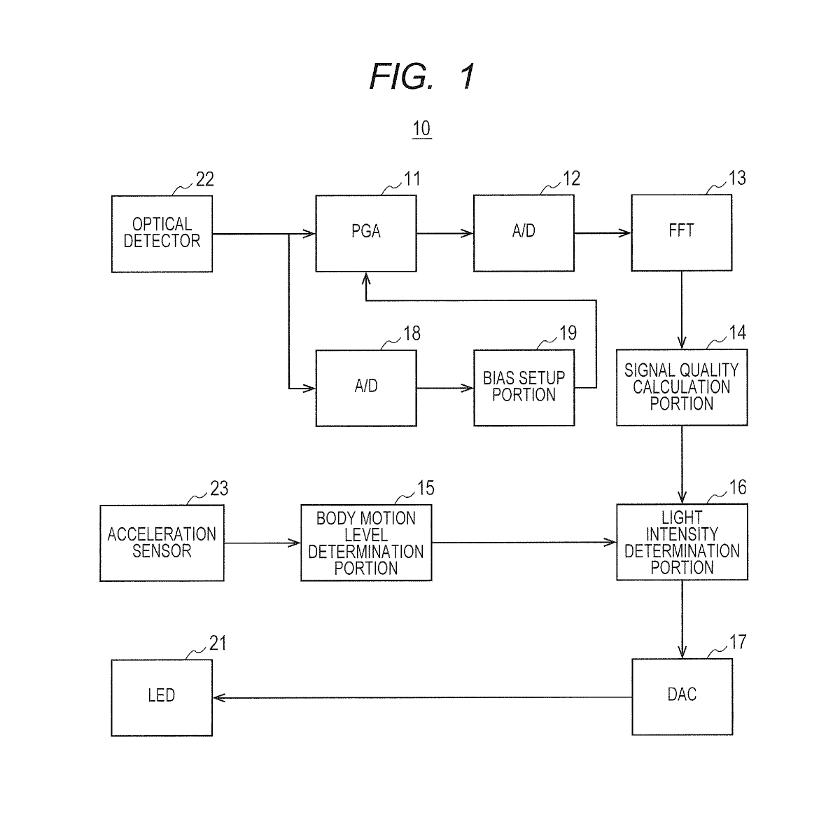

FIG. 1 illustrates a pulsation measuring apparatus according to an embodiment. A pulsation measuring apparatus 10 includes a PGA 11, an AD converter 12, an FFT (Fast Fourier Transform) portion 13, a signal quality calculation portion 14, a body motion level determination portion 15, a light intensity determination portion 16, a DAC (Digital to Analog Convertor) 17, an LED (Light Emitting Diode) 21, an optical detector 22, and an acceleration sensor 23. The pulsation measuring apparatus 10 is a wearable apparatus attached to a subject being tested (subject), for example. The pulsation measuring apparatus 10 is configured as a wristband-type apparatus, for example, and is attached to an arm or a wrist of a user. The pulsation measuring apparatus 10 is driven by a battery, for example.

The LED 21 configures a light-emitting portion and irradiates the light to a subject. The LED 21 irradiates the light to a measurement site where a blood vessel of the subject exists. The subject may be a human being or any animal other than a human being. The LED 21 cyclically irradiates pulsed light to the measurement site under control of an unshown controller, for example. A wavelength of the light irradiated from the LED 21 is appropriately selected according to measurement conditions.

The optical detector 22 receives the reflected light resulting from the light that is irradiated from the LED 21 and reflects off the subject. The optical detector 22 then outputs a detection signal (optical detection signal) for the reflected light. The optical detector 22 can use a phototransistor or a photodiode. The intensity of an optical detection signal output from the optical detector 22 varies with pulsing motion in a blood vessel. The LED 21 and the optical detector 22 are placed alongside at different positions on the same substrate, for example.

The PGA 11 amplifies an optical detection signal output from the optical detector 22. The PGA 11 is configured as a programmable instrumentation amplifier that can change gains, for example. The PGA 11 is configured to be capable of varying signal levels of optical detection signals input to the AD converter 12. The AD converter 12 converts an optical detection signal output from the optical detector 22 into a digital signal. The AD converter 12 uses a delta-sigma AD converter, for example. The FFT portion 13 applies fast Fourier transform to an optical detection signal converted into a digital value to generate a pulsation signal (pulse wave signal). The FFT portion 13 configures a pulsation signal generation portion.

The signal quality calculation portion 14 calculates the signal quality of a pulsation signal generated by the FFT portion 13. The signal quality calculation portion 14 calculates an SN ratio (Signal to Noise Ratio) of the pulsation signal, for example. The signal quality calculation portion 14 calculates an SN ratio by finding a ratio of areas corresponding to a peak part and the nearby part of a spectrum for the pulsation signal resulting from the fast Fourier transform, for example. The signal quality calculation portion 14 may calculate an SN ratio by finding a ratio of a DC component and an AC component in the pulsation signal.

The acceleration sensor 23 detects an acceleration of the subject. The acceleration sensor 23 is housed in a wristband-type apparatus configuring the pulsation measuring apparatus 10, for example. The body motion level determination portion (body motion determination portion) 15 determines whether the subject remains under quiescent conditions, based on the acceleration detected by the acceleration sensor 23.

The light intensity determination portion 16 controls the luminescence quantity of the LED 21. Control (adjustment) of the luminescence quantity for the LED 21 is hereinafter also referred to as optical calibration. The luminescence quantity of the LED 21 depends on the magnitude of a supplied electric current. The light intensity determination portion 16 controls the luminescence quantity of the LED 21 by controlling an electric current supplied to the LED 21. The light intensity determination portion 16 controls the luminescence quantity of the LED 21 within a predetermined range, for example.

After the pulsation measurement starts, the light intensity determination portion 16 determines the luminescence quantity (its control value) of the LED 21 based on the SN ratio calculated by the signal quality calculation portion 14. According to the embodiment, the light intensity determination portion 16 determines the luminescence quantity based on the SN ratio when the body motion level determination portion 15 determines that the subject remains under quiescent conditions after the pulsation measurement starts. The light intensity determination portion 16 determines the luminescence quantity when the subject is determined to remain under quiescent conditions for a specified time or longer, for example. The light intensity determination portion 16 decreases the luminescence quantity when the SN ratio is higher than or equal to threshold value 1, or increases the luminescence quantity when the SN ratio is lower than or equal to threshold value 2 that is smaller than threshold value 1, for example. The light intensity determination portion 16 controls an electric current supplied to the LED 21 via the DAC 17 that converts a digital value into an analog value.

The AD converter 18 converts an optical detection signal output from the optical detector 22 into a digital signal. The AD converter 18 uses a successive-approximation type AD converter, for example. A quantization bit rate for the AD converter 18 may be lower than a quantization bit rate for the AD converter 12. For example, the AD converter 12 uses an AD converter at the 24-bit quantization bit rate. The AD converter 18 uses an AD converter at the 10-bit quantization bit rate.

A bias setup portion 19 outputs a reference voltage to the PGA 11 and uses the reference voltage to control the signal level of a signal output from the PGA 11. The bias setup portion 19 determines the signal level variation of a signal output from the PGA 11 based on the digital signal converted by the AD converter 18. A value corresponding to the signal level variation in the PGA 11 is hereinafter also referred to as a bias value. The PGA 11 decreases the signal level of an optical detection signal input to the AD converter 12 by an amount corresponding to the reference voltage to vary the signal level of an optical detection signal input to the AD converter 12, for example. According to the embodiment, the light intensity determination portion 16 has a function of adjusting an electric current supplied to the LED 21 when the signal level variation in the PGA 11 reaches an upper limit or a lower limit.

In the pulsation measuring apparatus 10, the PGA 11, the AD converter 12, the DAC 17, and the AD converter 18 can be configured as hardware placed inside a microcomputer, for example. The microcomputer (analog microcomputer) includes a processor. The processor operates based on a program to be capable of implementing at least part of functions of the FFT portion 13, the signal quality calculation portion 14, the body motion level determination portion 15, the light intensity determination portion 16, and the bias setup portion 19.

### [Operation Procedure 1: Optical Calibration at the Start of Measurement]

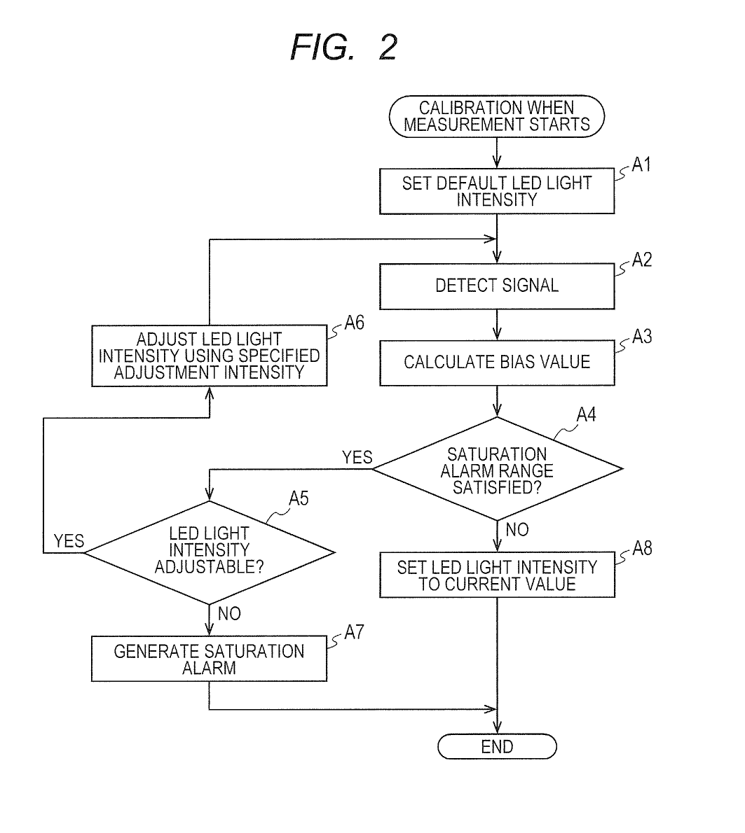

FIG. 2 illustrates an operation procedure for the optical calibration at the start of measurement. The optical calibration at the start of measurement is performed when the pulsation measuring apparatus 10 is turned on or the pulsation measurement starts on the pulsation measuring apparatus 10. The light intensity determination portion 16 sets the luminescence quantity (control value for the supply current magnitude) of the LED 21 to a specified value (default value) (step A1). At step A1, the light intensity determination portion 16 settles the specified value by using a center value between the maximum value and the minimum value for the luminescence quantity of the LED 21 in terms of control, for example. The light intensity determination portion 16 outputs the control value set at step A1 to the DAC 17. The DAC 17 converts the input control value into an analog voltage and applies the voltage to the LED 21. The LED 21 lights based on the luminescence quantity corresponding to the electric current supplied via the DAC 17.

After the LED 21 lights, the optical detector 22 detects the reflected light reflecting off a subject and outputs an optical detection signal (step A2). The AD converter 18 converts the optical detection signal into a digital signal. The bias setup portion 19 determines a signal level variation (bias value) in the PGA 11 based on the optical detection signal converted into the digital signal (step A3). The bias value here is assumed to equal a digital value for the AD-converted optical detection signal. The bias setup portion 19 varies the bias value corresponding to the magnitude of the optical detection signal detected at step A2. The signal level of the optical detection signal output from the PGA 11 can thereby fall within an input voltage range of the AD converter 12.

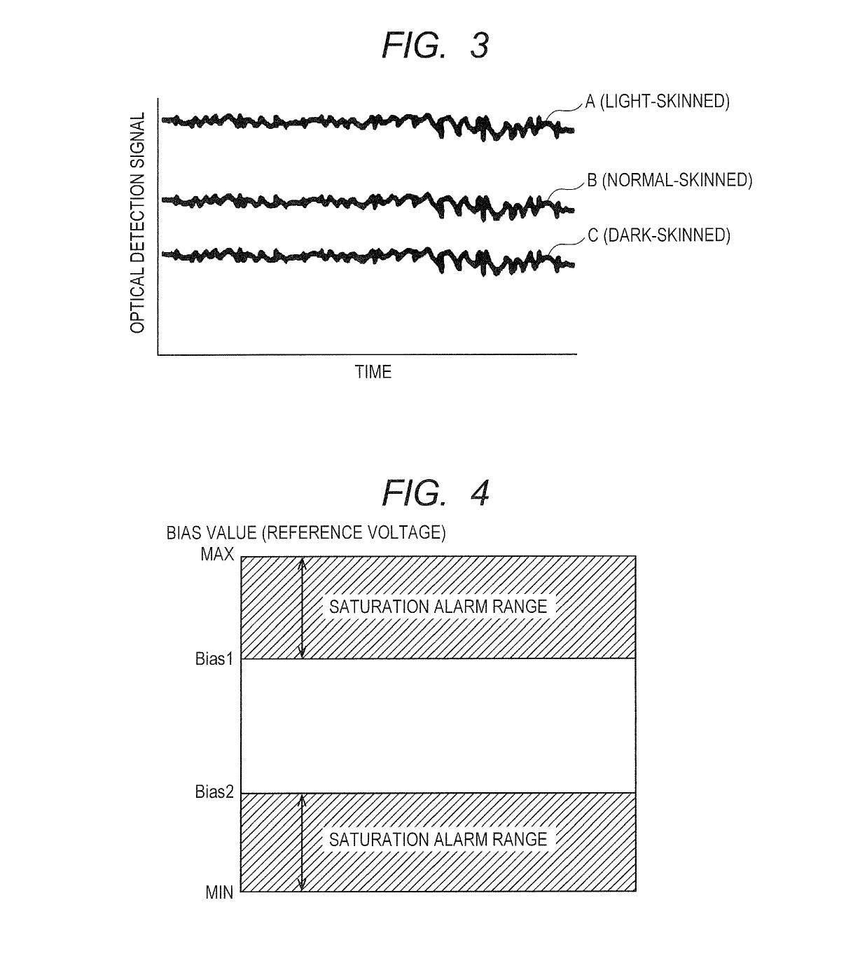

FIG. 3 illustrates example optical detection signals output from the optical detector 22. In FIG. 3, the horizontal axis represents the time and the vertical axis represents the magnitude of an optical detection signal. Graph A represents an optical detection signal when an untested area is relatively colored light, namely, when the skin color is relatively light (light-skinned). Graph B represents an optical detection signal when the skin color is normal (normal-skinned). Graph C represents an optical detection signal when the skin color is relatively dark (dark-skinned).

As illustrated in FIG. 3, the level of an optical detection signal output from the optical detector 22 varies with the untested area color. Intensifying the whiteness of the untested area increases a reflectance and increases the signal level of the optical detection signal. The bias setup portion 19 increases the bias value as the optical detection signal increases, thus increasing an amplitude of the signal level to be decreased in the PGA 11, for example. The bias setup portion 19 includes a DAC, for example, and converts a digital signal output from the AD converter 18 into an analog voltage (reference voltage). The PGA 11 decreases the signal level by an amount corresponding to the reference voltage, thereby making it possible to supply the AD converter 12 with the optical detection signal at a certain level independently of skin colors.

Returning to FIG. 2, the light intensity determination portion 16 determines whether the bias value determined at step A3 falls within a saturation alarm range (step A4). FIG. 4 illustrates an example of setting the saturation alarm range. The bias value ranges from a minimum value (MIN) to a maximum value (MAX) both predetermined. The light intensity determination portion 16 determines that the bias value falls within the saturation alarm range when the bias value falls within the range from maximum value MAX to Bias1 resulting from maximum value MAX minus a specified value. The light intensity determination portion 16 determines that the bias value falls within the saturation alarm range when the bias value falls within the range from minimum value MIN to Bias2 resulting from minimum value MIN plus a specified value. The light intensity determination portion 16 determines that the bias value falls outside the saturation alarm range when the bias value falls within the range from Bias1 to Bias2.

The light intensity determination portion 16 may determine at step A4 that the bias value falls within the saturation alarm range, and then determines whether the luminescence quantity of the LED 21 is adjustable (step A5). The signal level of a detection signal needs to be decreased by decreasing the luminescence quantity of the LED 21 when the signal level of the optical detection signal is high and the bias value is therefore set to be large. Contrarily, the signal level of a detection signal needs to be increased by increasing the luminescence quantity of the LED 21 when the signal level of the optical detection signal is low and the bias value is therefore set to be small. At step A5, the light intensity determination portion 16 determines whether the luminescence quantity of the LED 21 can be adjusted as intended.

Specifically at step A5, the light intensity determination portion 16 determines whether the current luminescence quantity (supply current) of the LED 21 equals the minimum value in terms of control when the bias value falls within the range between Bias1 and MAX in FIG. 4, for example. The light intensity determination portion 16 determines that the luminescence quantity can be adjusted when the luminescence quantity is not the minimum value, or determines that the luminescence quantity cannot be adjusted when the luminescence quantity equals the minimum value. The light intensity determination portion 16 determines whether the current luminescence quantity of the LED 21 equals the maximum value in terms of control when the bias value falls within the range between Bias2 and MIN in FIG. 4, for example. The light intensity determination portion 16 determines that the luminescence quantity can be adjusted when the luminescence quantity is not the maximum value, or determines that the luminescence quantity cannot be adjusted when the luminescence quantity equals the maximum value.

When the luminescence quantity is determined to be adjustable at step A5, the light intensity determination portion 16 increases or decreases the luminescence quantity of the LED 21 by a specified adjustment amount (step A6). After the luminescence quantity is adjusted, the LED 21 lights based on the adjusted luminescence quantity. The process then returns to step A2 to detect an optical detection signal. When the luminescence quantity is determined to be not adjustable at step A5, an unshown lamp or motor is driven, for example. A user is notified of a saturation alarm by light or vibration (step A7). The process then terminates.

A loop including steps A2 through A6 is repeated until the bias value is determined to not fall within the saturation alarm range at step A4. When determining at step A4 that the bias value does not fall within the saturation alarm range, the light intensity determination portion 16 sets the luminescence quantity of the LED 21 to the most recently set (adjusted) luminescence quantity (step A8). The luminescence quantity set at step A8 is used as an initial setting for the pulsation measurement to be performed subsequently. The FFT portion 13 may or may not generate a pulsation signal during the optical calibration at the start of measurement.

Suppose the luminescence quantity of the LED 21 is set to the maximum value of 2233 for the DAC 17 in terms of control at step A1 and the luminescence quantity of the LED 21 is adjusted based on a tolerance of ±100 at step A6. The above-mentioned loop is assumed to cycle in units of 117 [ms], for example. In this case, assume that the finally adjusted luminescence quantity of the LED 21 equals the minimum value of 435 for the DAC 17 in terms of control. The loop is then repeated 18 times until the adjustment of the luminescence quantity is completed. This case causes the maximum number of loops and requires approximately two seconds for the adjustment. The number of loops is halved when the initial luminescence quantity of the LED 21 equals the center value between the maximum value and the minimum value. The time required for the adjustment approximates to one second. The optical calibration at the start of measurement can be performed fast because there is no need to use an output signal from the delta-sigma AD converter 12.

### [Operation Procedure 2: Optical Calibration During Measurement]

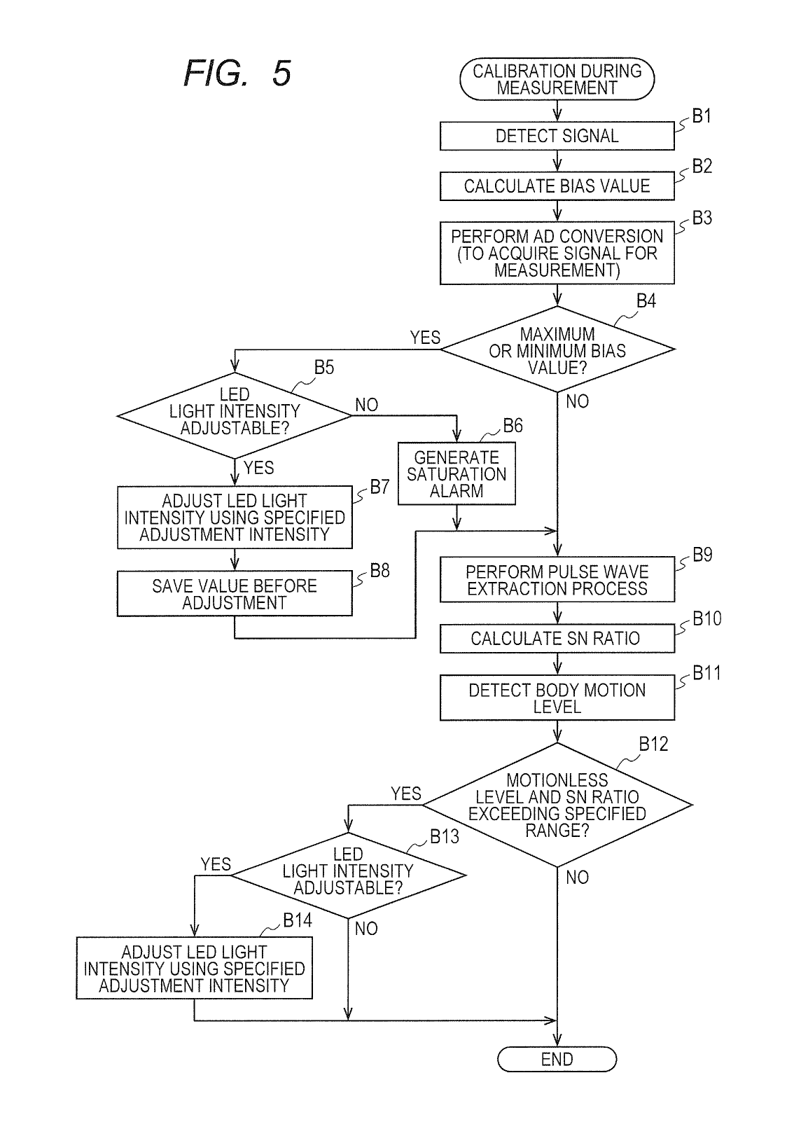

FIG. 5 illustrates an operation procedure for the optical calibration during pulsation measurement. After the LED 21 lights, the optical detector 22 detects the reflected light reflecting off a subject and outputs an optical detection signal (step B1). The AD converter 18 AD-converts the optical detection signal. The bias setup portion 19 determines a bias value based on the optical detection signal (step B2). In this case, the bias value is determined similarly to step A3 in FIG. 2. The bias value determined at step B2 is used for the reference voltage for the PGA 11 to detect an optical detection signal next time.

Concurrently with step B2, the AD converter 12 converts the optical detection signal input via the PGA 11 into a digital signal (step B3). The AD converter 12 converts the optical detection signal into the digital signal and thereby generates the signal used for the measurement.

The light intensity determination portion 16 determines whether the bias value determined at step B2 equals the maximum value or the minimum value (step B4). The light intensity determination portion 16 may determine at step B4 that the bias value equals the maximum value or the minimum value, and then adjusts the luminescence quantity of the LED 21 (luminescence quantity adjustment 1).

The light intensity determination portion 16 may determine at step B4 that the bias value equals the maximum value or the minimum value, and then determines whether the luminescence quantity of the LED 21 can be adjusted (step B5). At step B5, the light intensity determination portion 16 determines whether the luminescence quantity of the LED 21 can be adjusted as intended.

Specifically at step B5, the light intensity determination portion 16 determines whether the current luminescence quantity of the LED 21 equals the minimum value in terms of control when the bias value equals maximum value MAX (see FIG. 4), for example. The light intensity determination portion 16 determines that the luminescence quantity can be adjusted when the luminescence quantity is not the minimum value, or determines that the luminescence quantity cannot be adjusted when the luminescence quantity equals the minimum value. The light intensity determination portion 16 determines whether the current luminescence quantity of the LED 21 equals the maximum value in terms of control when the bias value equals minimum value MIN, for example. The light intensity determination portion 16 determines that the luminescence quantity can be adjusted when the luminescence quantity is not the maximum value, or determines that the luminescence quantity cannot be adjusted when the luminescence quantity equals the maximum value.

When the luminescence quantity is determined to be adjustable at step B5, the light intensity determination portion 16 increases or decreases the luminescence quantity of the LED 21 by a specified adjustment amount (step B7). The light intensity determination portion 16 saves the luminescence quantity of the LED 21 before the luminescence quantity is increased or decreased (step B8). When the luminescence quantity is determined to be not adjustable at step B5, an unshown lamp or motor is driven, for example. A user is notified of a saturation alarm by light or vibration (step B6). LED luminescence quantity adjustment 1 terminates according to the above-mentioned procedure.

A pulse wave extraction process is performed on the optical detection signal converted into the digital value at step B3 (step B9) when the bias value is determined to be not the maximum value or the minimum value at step B4 or when the above-mentioned luminescence quantity adjustment 1 terminates. The pulse wave extraction process at step B9 includes FFT performed by the FFT portion 13 on an optical detection signal. The signal quality calculation portion 14 calculates an SN ratio of the pulse wave signal generated from FFT performed by the FFT portion 13 (step B10).

Concurrently with the above-mentioned procedure, the body motion level determination portion 15 determines a body motion level based on a signal input from the acceleration sensor 23 (step B11). The light intensity determination portion 16 determines whether the body motion level determined at step B11 indicates the quiescent state of a subject and the SN ratio calculated at step B10 falls outside a specified range (step B12).

At step B12, the light intensity determination portion 16 determines whether the quiescent state of the subject continues for a specified time or longer and the SN ratio is higher than or equal to threshold value 1 or is smaller than or equal to threshold value 2. The light intensity determination portion 16 may determine that the subject is quiescent and the SN ratio falls outside the specified range, and then adjusts the light intensity of the LED 21 (LED light intensity adjustment 2). The light intensity determination portion 16 performs LED light intensity adjustment 2 to decrease the luminescence quantity of the LED 21 when the SN ratio is higher than or equal to threshold value 1, or increase the luminescence quantity of the LED 21 when the SN ratio is lower than or equal to threshold value 2.

The light intensity determination portion 16 may determine at step B12 that the subject is quiescent and the SN ratio falls outside the specified range, and then determines whether the luminescence quantity of the LED 21 can be adjusted (step B13). At step B13, the light intensity determination portion 16 determines whether the luminescence quantity of the LED 21 can be adjusted as intended. At step B13, the light intensity determination portion 16 uses the luminescence quantity (its control value) saved at step B8 as a saturation limit value and determines that the luminescence quantity cannot be adjusted when the luminescence quantity is greater or smaller than or equal to the saturation limit value.

Specifically, when the SN ratio is higher than or equal to threshold value 1, the light intensity determination portion 16 determines whether the current luminescence quantity of the LED 21 equals the minimum value in terms of control or is smaller than or equal to the saturation limit value. The light intensity determination portion 16 determines that the luminescence quantity can be adjusted when the luminescence quantity is not the minimum value or is not smaller than or equal to the saturation limit value, or determines that the luminescence quantity cannot be adjusted when the luminescence quantity equals the minimum value or is smaller than or equal to the saturation limit value. When the SN ratio is lower than or equal to threshold value 2, the light intensity determination portion 16 determines whether the current luminescence quantity of the LED 21 equals the maximum value in terms of control or is greater than or equal to the saturation limit value. The light intensity determination portion 16 determines that the luminescence quantity can be adjusted when the luminescence quantity is not the minimum value or is not greater than or equal to the saturation limit value, or determines that the luminescence quantity cannot be adjusted when the luminescence quantity equals the maximum value or is greater than or equal to the saturation limit value. When the luminescence quantity is determined to be adjustable at step B13, the light intensity determination portion 16 increases or decreases the luminescence quantity of the LED 21 by a specified adjustment amount (step B14).

As above, the value saved at step B8 is used as the saturation limit value for the following reason. Suppose LED luminescence quantity adjustment 2 is performed even though the luminescence quantity of the LED 21 is larger or smaller than or equal to the luminescence quantity saved at step B8. LED luminescence quantity adjustment 2 adjusts the luminescence quantity so as to cause the saturation more easily than the state before the luminescence quantity is once adjusted to hardly cause the saturation, though. In this case, LED luminescence quantity adjustment 1 is required again. LED luminescence quantity adjustment 1 and LED luminescence quantity adjustment 2 are performed repeatedly. The value before the adjustment is saved at step B8 and is used as a value (saturation limit value) that, if exceeded, causes the saturation when the luminescence quantity is adjusted. This can prevent LED luminescence quantity adjustment 1 and LED luminescence quantity adjustment 2 from looping.

The above-mentioned procedure is performed each time the LED 21 lights, for example. The luminescence quantity of the LED 21 is adjusted during the pulsation measurement.

### [Operating Waveform Examples]

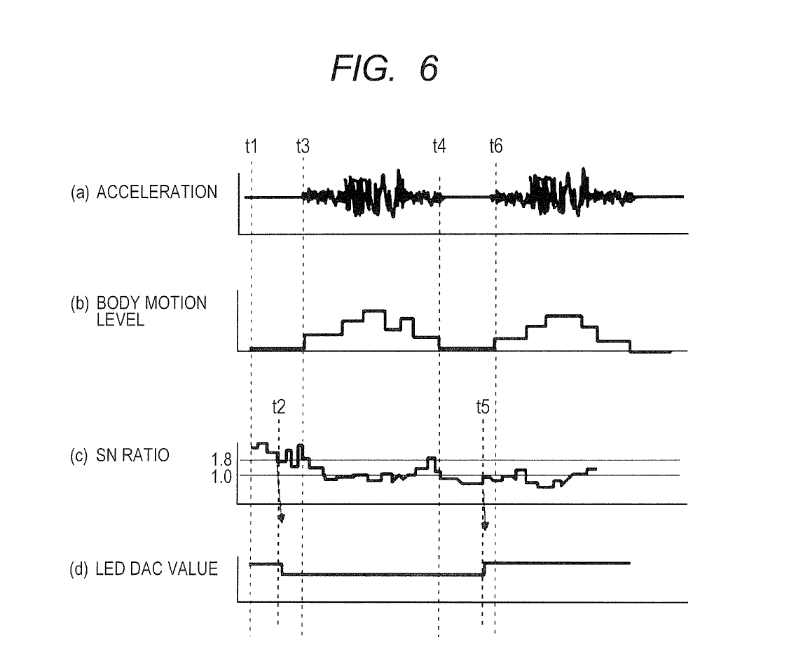

FIG. 6 illustrates operating waveforms of the respective portions during pulsation measurement. The acceleration sensor 23 detects motion (acceleration) of a subject while the pulsation measuring apparatus 10 operates. Decreasing the subject motion decreases the amplitude of an output signal from the acceleration sensor 23 and increasing the subject motion increases the amplitude thereof (see (a)). The output signal from the acceleration sensor 23 varies from hour to hour depending on active states of the subject.

The body motion level determination portion 15 determines a body motion level of the subject by applying a threshold value process to the amplitude of the output signal from the acceleration sensor 23, for example. The body motion level determination portion 15 determines whether the body motion level of the subject corresponds to quiescence (level 0) or otherwise (see (b)), based on the amplitude of the output signal from the acceleration sensor 23.

The signal quality calculation portion 14 calculates the SN ratio of a pulsation signal each time the LED 21 lights to generate the pulsation signal, for example. The pulsation signal SN ratio calculated by the signal quality calculation portion 14 can vary with the luminescence quantity of the LED 21 or active states of the subject (see (c)). The SN ratio here is used as a determination criterion to determine whether to perform the above-mentioned LED luminescence quantity adjustment 2 and uses threshold value 1 assumed to be “1.8” and threshold value 2 assumed to be “1.0”.

Suppose the subject enters the quiescent state at time t1 and the state of body motion level 0 continues five seconds, for example. With reference to (c) in FIG. 6, the SN ratio exceeds “1.8” even at the time point slightly later than time t1. In this case, the light intensity determination portion 16 assumes the SN ratio to be excessive and allows the DAC 17 to decrease the luminescence quantity of the LED 21 at time t2 (see (d)). The voltage output from the DAC 17 decreases and the luminescence quantity of the LED 21 decreases to be capable of preventing the pulsation measuring apparatus 10 from being used at an unnecessarily high SN ratio and preventing a battery from being drained unnecessarily.

Suppose the subject starts to be active at time t3 to cause the body motion level to be higher than 0. In this case, the light intensity determination portion 16 does not adjust the luminescence quantity of the LED 21 even if the SN ratio is lower than or equal to “1.0.” Suppose the subject comes to be quiescent at time t4 and the state of body motion level 0 continues five seconds or longer. The SN ratio is lower than “1.0” at time t5. The light intensity determination portion 16 therefore assumes the SN ratio to be lowered and allows the DAC 17 to increase the luminescence quantity of the LED 21 (see (d)). Increasing the luminescence quantity of the LED 21 increases the signal level of the optical detection signal output from the optical detector 22. The SN ratio can be expected to increase.

Suppose the subject starts to be active at time t6 to cause the body motion level to be higher than 0. In this case, the light intensity determination portion 16 does not adjust the luminescence quantity of the LED 21 even if the SN ratio is lower than or equal to “1.0.” The pulsation measuring apparatus 10 adjusts the luminescence quantity of the LED 21 when both conditions are satisfied, namely, the condition of the body motion level set to 0 and the condition of the SN ratio falling outside the range from “1.0” to “1.8”.

### [Overview]

According to the present embodiment, the body motion level determination portion 15 determines whether a subject remains under quiescent conditions, based on the information acquired from the acceleration sensor 23. The signal quality calculation portion 14 calculates the signal quality of a pulsation signal. The light intensity determination portion 16 controls the luminescence quantity of the LED 21 based on the signal quality calculated by the signal quality calculation portion 14 when the body motion level determination portion 15 determines the subject to be quiescent. The present embodiment can adjust the luminescence quantity of the LED 21 while the pulsation is measured. The luminescence quantity can be adjusted correspondingly to a change, if any, in measurement situations after the measurement starts.

According to the present embodiment, the light intensity determination portion 16 increases the luminescence quantity of the LED 21 when the subject remains quiescent and the signal quality (SN ratio) of a pulsation signal is lower than or equal to threshold value 2. The luminescence quantity of the LED 21 is then increased. More intense light is thereby irradiated to the subject. Consequently, it is possible to increase pulse wave components in an optical detection signal detected by the optical detector 22. The light intensity determination portion 16 decreases the luminescence quantity of the LED 21 when the subject remains quiescent and the signal quality of a pulse wave signal is higher than or equal to threshold value 1. In this case, the pulsation measuring apparatus 10 can be prevented from being used while the pulsation signal quality remains unnecessarily high. Decreasing the luminescence quantity of the LED 21 can reduce the power consumption.

Suppose the luminescence quantity of the LED 21 is adjusted only under the condition that the subject remains quiescent. It is then impossible to determine whether to increase or decrease the luminescence quantity of the LED 21. Suppose the luminescence quantity of the LED 21 is adjusted only under the condition of the pulsation signal quality. The luminescence quantity is then adjusted based on a pulsation signal even when the subject is not quiescent. Generally, when the subject is not quiescent, the pulsation signal quality degrades compared to when the subject is quiescent. The control over the luminescence quantity of the LED 21 is likely to adjust the luminescence quantity so as only to increase the luminescence quantity. In this case, a sensing region is eventually exceeded to risk causing saturation.

The present embodiment combines determination on the quiescent state of a subject using the acceleration sensor 23 with observation on the signal quality calculated by the signal quality calculation portion 14. The combination can appropriately adjust the luminescence quantity of the LED 21 even during the measurement of pulsation in the pulsation measuring apparatus 10. Appropriately setting the luminescence quantity of the LED 21 can reduce the power consumption without degrading the measurement accuracy.

According to the present embodiment, the bias setup portion 19 determines a reference voltage input to the PGA 11 based on the optical detection signal converted into a digital signal by using the AD converter 18. The present embodiment can compensate for reflectance differences due to individual differences by varying the reference voltage according to the signal level of the optical detection signal and can ensure an appropriate signal level for the optical detection signal input to the AD converter 12. The reference voltage can be directly set, making it possible to shorten the time required to adjust the luminescence quantity of the LED 21. The microcomputer may include the delta-sigma AD converter used for measurement and also a sequential-comparison AD converter whose quantization bit rate is lower than that of the delta-sigma AD converter. In this case, the delta-sigma AD converter can be used to generate pulse wave signals and the sequential-comparison AD converter can be used to set the reference voltage for the PGA 11 to be capable of setting an appropriate reference voltage without needing for additional resources.

While there has been described the specific embodiment of the invention made by the inventors, it is to be distinctly understood that the present invention is not limited to the above-mentioned embodiment and may be embodied in various modifications without departing from the spirit and scope of the invention.

What is claimed is:

1. A pulsation measuring apparatus comprising:
a light-emitting portion that irradiates light to a subject;
an optical detector that detects light reflecting off the subject and outputs an optical detection signal;
a pulsation signal generation portion that generates a pulsation signal based on the optical detection signal;
a signal quality calculation portion that calculates signal quality of the pulsation signal;
an acceleration sensor that detects acceleration of the subject;
a body motion determination portion that determines whether the subject maintains a quiescent state based on acceleration detected by the acceleration sensor, and
a luminescence quantity controller that controls luminescence quantity of the light-emitting portion based on the signal quality when the body motion determination portion determines that a subject maintains a quiescent state.

2. The pulsation measuring apparatus according to claim 1,
wherein the luminescence quantity controller decreases the luminescence quantity when the signal quality is higher than or equal to a first threshold value, and increases the luminescence quantity when the signal quality is lower than or equal to a second threshold value that is smaller than a first threshold value.

3. The pulsation measuring apparatus according to claim 1, further comprising:
a first analog-digital converter that converts the optical detection signal into a digital signal and output the converted digital signal to the pulsation signal generation portion.

4. The pulsation measuring apparatus according to claim 3,
wherein the first analog-digital converter is a delta-sigma analog-digital converter.

5. The pulsation measuring apparatus according to claim 3, further comprising:
a programmable gain amplifier configured to be capable of varying a signal level of an optical detection signal input to the first analog-digital converter;
a second analog-digital converter that converts the optical detection signal into a digital signal; and
a bias setup portion that determines a signal level variation of an optical detection signal in the programmable gain amplifier based on a digital signal converted by the second analog-digital converter.

6. The pulsation measuring apparatus according to claim 5,
wherein a quantization bit rate of the first analog-digital converter is higher than a quantization bit rate of the second analog-digital converter.

7. The pulsation measuring apparatus according to claim 5,
wherein the luminescence quantity controller adjusts luminescence quantity of the light-emitting portion when a signal level variation in the programmable gain amplifier corresponds to one of an upper limit and a lower limit.

8. The pulsation measuring apparatus according to claim 7,
wherein the luminescence quantity controller saves a control value for the luminescence quantity before performing the adjustment, controls the luminescence quantity to decrease the luminescence quantity when a current control value for the luminescence quantity is larger than the saved control value, and controls the luminescence quantity to increase the luminescence quantity when a current control value for the luminescence quantity is smaller than the saved control value.

9. The pulsation measuring apparatus according to claim 1,
the luminescence quantity controller controls the luminescence quantity when the quiescent state continues longer than or equal to a specified time.

10. A light intensity control method in a pulsation measuring apparatus comprising the steps of:
irradiating light to a subject;
detecting reflected light resulting from the light reflecting off the subject;
generating a pulsation signal based on a detection signal for the reflected light;
calculating signal quality of the pulsation signal;
detecting acceleration of the subject by using an acceleration sensor and determining whether the subject maintains a quiescent state based on the detected acceleration, and
controlling light intensity of light irradiated to a subject based on the signal quality when the subject is determined to maintain a quiescent state.

11. A program that allows a processor to perform the steps of:
irradiating light to a subject;
generating a pulsation signal based on a detection signal for reflected light reflecting off the subject;
calculating signal quality of the pulsation signal;
determining whether the subject maintains a quiescent state based on acceleration of the subject detected by using an acceleration sensor, and
controlling light intensity of light irradiated to a subject based on the signal quality when the subject is determined to maintain a quiescent state.
